(19) United States
(12) Patent Application Publication
(10) Pub. No.: US 2017/0273923 A1
(43) Pub. Date: Sep. 28, 2017

(54) METHOD OF ADMINISTERING DIVALPROEX

(71) Applicant: Sun Pharmaceutical Industries Ltd., Mumbai (IN)

(72) Inventors: Vishal Suhagiya, Baroda (IN); T. Nathamani, Baroda (IN); Amol Kulkarni, Baroda (IN); Shirish Kulkarni, Baroda (IN); Rajamannar Thennati, Baroda (IN)

(73) Assignee: Sun Pharmaceutical Industries Ltd., Mumbai (IN)

(21) Appl. No.: 15/467,580

(22) Filed: Mar. 23, 2017

(30) Foreign Application Priority Data
Mar. 23, 2016 (IN) 201621010256

(51) Int. Cl. A61K 31/19; A61K 9/28; A61K 9/20

(52) U.S. Cl. CPC A61K 31/19; A61K 9/2072; A61K 9/2054; A61K 9/2027; A61K 9/2009; A61K 9/2013; A61K 9/2866

(57) ABSTRACT

A compact sustained release tablet comprising divalproex or its pharmaceutically acceptable salts as a sole active ingredient is present in an amount equivalent to from about 700 mg to about 1500 mg of valproic acid and pharmaceutically acceptable tablet excipients; wherein weight ratio of pharmaceutically acceptable tablet excipients to divalproex and/or its salt is less than 1, the tablet is suitable for once a day administration and when orally administered, with or without food, the ratio of mean AUC₀_{_}_{inf-fasted} to mean AUC₀_{_}_{inf-fed} is within the range of 0.9 to 1.1.

### FIELD OF THE INVENTION

The present invention relates to compact sustained release tablets of divalproex, which are suitable for once a day administration.

### BACKGROUND OF THE INVENTION

Valproic acid and its derivatives are indicated for the treatment of different types of epilepsy, for the treatment of acute manic or mixed episodes associated with bipolar disorder, and for prophylaxis of migraine headaches. Particularly, a complex constituted by a 1:1 molar relationship of valproic acid and sodium valproate (divalproex sodium) has recently reached some preference in the market. Divalproex sodium is marketed by Abbott Laboratories as conventional delayed-release (DR), enteric-coated, dosage forms (Depakote® tablets, and Depakote™ sprinkle capsules), and as innovative extended release (ER) tablets (Depakote™ ER). Both tablets (divalproex-DR and divalproex-ER) are supplied in dosage strengths containing divalproex sodium equivalent to 250 and 500 mg of valproic acid. The main problem with these strengths of marketed divalproex extended release tablets Depakote™ ER is the large number of pills that need to be administered to a patient. For example, the recommended initial daily dose for the treatment of mania is 25 mg/kg/day. Assuming a patient has a 75 kg body weight, the daily dose would be 1875 mg, for which the patient is required to ingest three units of the 500 mg divalproex extended release tablet and one unit of the 250 mg divalproex extended release tablet available under the brand name of Depakote™ ER. Further according to prescription label for Depakote™ ER, this dose is increased as rapidly as possible to achieve therapeutic response or desired plasma levels. Therefore the number of tablets increase but when the dose is stabilised, the patient is still required to ingest several tablets. If the patient is stabilised at the maximum allowable dose of 60 mg/kg/day and weighs about 66 kg, then he will require a dose of 4000 mg/day. Thus he will be required to ingest 8 tablets of Depakote™ ER 500 mg.

For the treatment of epilepsy (Complex partial seizures and absence seizures), the recommended daily dose is 15 mg/kg/day. Assuming a 75 kg body weight patient, the daily dose would be 1125 mg. Therefore for a initial dose of 1125 mg the patient is required to ingest two units of 500 mg and one unit of 250 mg for an initial daily dose of 1250 mg. This dose is increased on a weekly basis. If the patient is stabilised at the maximum allowable dose of 60 mg/kg/day and weighs about 66 kg, then he will require a dose of 4000 mg/day. Thus he will be required to ingest eight tablets of Depakote™ ER 500 mg. Ingestion of multiple pills is inconvenient for a patient suffering from mania or epilepsy and particularly more so if the tablets are large in size.

Hence, first there is need for a formulation of higher strength of divalproex in the range from 700 to 1500 mg to reduce the psychological impact of ingesting a large number of lower strength tablets. Secondly, as the larger strength tablets at the same level of excipients will be difficult to swallow there is a need for compact tablets of higher strengths with lower level of excipients. More importantly, with these limitations imposed on the compact tablets there is a need that the compact sustained release tablets provide consistent blood levels that remain within the narrow therapeutic window for divalproex. The present inventors have found such compact sustained release tablets of divalproex. More particularly, the present inventors have found compact sustained release tablet comprising divalproex or its pharmaceutically acceptable salts as a sole active ingredient is present in an amount equivalent to from about 700 mg to about 1500 mg of valproic acid and pharmaceutically acceptable tablet excipients; wherein weight ratio of pharmaceutically acceptable tablet excipients to divalproex and/or its salt is less than 1, the tablet is suitable for once a day administration and when orally administered, with or without food, the ratio of mean AUC_{0 inf-fasted} to mean AUC_{0 inf-fed} is within the range of 0.9 to 1.1.

### SUMMARY OF THE INVENTION

The present invention provides a compact sustained release tablet comprising divalproex or its pharmaceutically acceptable salts as a sole active ingredient present in an amount equivalent to from about 700 mg to about 1500 mg of valproic acid and pharmaceutically acceptable tablet excipients; wherein weight ratio of pharmaceutically acceptable tablet excipients to divalproex and/or its salt is less than 1, the tablet is suitable for once a day administration and when orally administered, with or without food, the ratio of mean AUC₀_{_}_{inf-fasted} to mean AUC₀_{_}_{in-fed} is within the range of 0.9 to 1.1.

### BRIEF DESCRIPTION OF THE DRAWINGS

FIG. 1: In vitro dissolution of compact sustained release tablet of divalproex 1000 mg prepared as of example 1.

FIG. 2: Mean plasma concentration vs time profile of valproic acid (μg/ml) obtained upon oral administration of compact sustained release tablet having 1000 mg of valproic acid equivalent amount of Divalproex, when administered in fed and fasted state.

### DESCRIPTION OF THE INVENTION

The term ‘compact tablet’ means the tablet is of swallowable size. For example, the total weight of the tablet is at the most 1900 mg, more preferably it is less than 1500 mg. The ratio of the weight of pharmaceutical excipients to divalproex and/or its salt is less than 1. Preferably the ratio is less than 0.8, more preferably less than 0.6 and most preferably less than 0.4.

The term mean AUC₀_{_}_{inf-fasted} as used herein means the area under the mean plasma concentration vs time curve, achieved upon administration of the compact sustained release tablet of the present invention in fasted state or without food.

The term mean AUC₀_{_}_{inf-fed} as used herein means the area under the mean plasma concentration vs time curve, achieved upon administration of the compact sustained release tablet of the present invention in fed state or with food.

According to the present invention, the ratio of mean AUC₀_{_}_{inf-fasted} to mean AUC₀_{_}_{inf-fed} is within the range of 0.9 to 1.1. It can be 0.9, 0.95, 0.99, 1.0, 1.05, 1.1 and so on. In one preferred embodiment, the ratio of mean AUC₀_{_}_{inf-fasted} to mean AUC₀_{_}_{inf-fed} is 1.04. This means that the oral bioavailability is substantially equivalent, when the compact sustained release tablet of the present invention are administered in the fasted state i.e without food or in the fed state i.e with food.

In one aspect, the present invention also provides a compact sustained release tablet comprising divalproex or its pharmaceutically acceptable salts as a sole active ingredient present in an amount equivalent to from about 700 mg to about 1500 mg of valproic acid, hydrophobic polymer and pharmaceutically acceptable tablet excipients; wherein the weight ratio of pharmaceutically acceptable tablet excipients to divalproex or its salt is less than 1 and the tablet is suitable for once a day administration and when orally administered, with or without food, the ratio of mean AUC₀_{_}_{inf-fasted} to mean AUC₀_{_}_{inf-fed} is within the range of 0.9 to 1.1. Particularly, the compact sustained release tablet of the present invention, when the tablet is subjected to dissolution in 500 ml of 0.1N hydrochloric acid for 45 min followed by 900 ml of pH 5.5 phosphate buffer with sodium lauryl sulphate using USP II dissolution apparatus rotating at a speed of 100 rotations per minute, releases divalproex or its pharmaceutically acceptable salts as follows:

| | % divalproex released | |
|---|---|---|
| Time in hours | Not Less than | Not more than |
| 3 | 10 | 20 |
| 9 | 40 | 50 |
| 12 | 50 | 60 |

Specifically, in one embodiment, the present invention provides a compact sustained release tablets comprising:
a hygroscopic active ingredient consisting of divalproex or its pharmaceutically acceptable salt as a sole active ingredient and pharmaceutically acceptable excipients wherein the tablet comprises
   a granular phase consisting essentially of a granulated mixture of divalproex or its pharmaceutically acceptable salts as a sole active ingredient, in an amount equivalent to from about 700 mg to about 1500 mg of valproic acid and a hydrophobic polymer; and
   an extragranular phase,
      wherein the weight ratio of pharmaceutical excipients to divalproex or its salt is less than 1, wherein the sustained release tablet gradually releases the divalproex over a period of 24 hours.

In one aspect, the present invention provides an oral compact sustained release tablet suitable for once a day, tablet comprising: a hygroscopic active ingredient consisting of divalproex or its pharmaceutically acceptable salt and pharmaceutically acceptable tablet excipients wherein the tablet comprises
a granular phase consisting essentially of a granulated mixture of divalproex or its pharmaceutically acceptable salts as a sole active ingredient in an amount equivalent to from about 700 mg to about 1500 mg of valproic acid and a hydrophobic polymer; and
an extragranular phase,
   wherein the weight ratio of pharmaceutically acceptable tablet excipients to divalproex and/or its salt is less than 1 and wherein when the tablet is subjected to dissolution in 500 ml of 0.1N HCl for 45 min followed by 900 ml of pH 5.5 phosphate buffer with sodium lauryl sulphate using USP II dissolution apparatus and 100 rotation per minute, at 37° C.±0.5° C., releases divalproex or its pharmaceutically acceptable salts as follows:

| | % divalproex released | |
|---|---|---|
| Time in hours | Not Less than | Not more than |
| 3 | 10 | 20 |
| 9 | 40 | 50 |
| 12 | 50 | 60 |

The granular phase consists essentially of a granulated admixture of divalproex or its pharmaceutically acceptable salts in an amount equivalent to from about 700 mg to about 1500 mg of valproic acid and a hydrophobic polymer and an extragranular phase. Apart from the hydrophobic polymer, the tablet contains other pharmaceutically acceptable excipients such as hydrophilic polymers, hydrophobic excipients, diluents, lubricants, binders and any other inactive excipients. In one specific embodiment, the compact sustained release tablet comprises hydrophilic polymer and hydrophobic polymer and are present in amount ranging from 25% to 50%, preferably, 30% to 45% by weight of the tablet.

Suitable examples of hydrophobic polymer (water insoluble polymer) include, but are not limited to, waxes, water insoluble cellulose derivatives such as ethyl cellulose, cellulose acetate, cellulose butyrate, insoluble grades of polyacrylates such as methacrylates, acrylic acid copolymers, high molecular weight polyvinyl alcohols etc. The divalproex or its salt is intimately mixed with a hydrophobic polymer such as ethyl cellulose. In one embodiment, ethyl cellulose is present in the range of about 5% to 10% of the weight of the tablet. The divalproex or its salt is intimately mixed with a hydrophobic polymer such as ethyl cellulose. In such embodiments, the granular phase “consists essentially of” divalproex or its pharmaceutically acceptable salts and hydrophobic polymer. This means that the granular phase does not contain other excipients that can interfere in controlling the hygroscopicity of the divalproex or its pharmaceutically acceptable salt and/or increase the bulk of the single unit of the compact sustained release tablet. The hydrophobic polymer also has a role as to control the dissolution rate and slows the rate of release of divalproex. Particularly, the hydrophobic polymer is admixed with divalproex or its salt, the amount of the hydrophobic polymer ranges from 1% to 25% by weight of the tablet, more preferably in the range of 3% to 20% by weight of the tablet and most preferably in the range of 5% to 15% by weight of the tablet. In an embodiment, the weight ratio of divalproex or its pharmaceutically acceptable salts to hydrophobic polymer present in the compact sustained release tablet of the present invention is about 10.

The granular phase in the compact sustained release tablet of the present invention is covered by an outer solid phase which is also referred herein to an extragranular phase. In one specific embodiment, the extragranular phase comprises a mixture of two or more hydrophilic polymer. The hydrophilic polymers are hydroxypropyl methyl cellulose having a molecular weight of 10000-1500000 and polyacrylic acid having a molecular weight of 700000 to 4000000 and viscosity of 3000 to 60000. In such embodiments, hydroxypropyl methyl cellulose is present in a range of about 20% to 40% by weight of the tablet and polyacrylic acid is present in the range of 0.5% to 1.0% by weight of the tablet.

Suitable examples of the hydrophilic polymer used in the compact sustained release tablet composition, include, but are not limited to, gelling type of polymers like polyacrylic acid (carbomer), gums such as xanthan gum, guar gum, alginate and the like. A number of different carbomer grades are commercially available that vary in their molecular weight, degree of crosslinking polymer structure and residual components. These differences account for the specific rheological handling and use characteristics of each grade. Carbomers designated with the letter ‘P’, eg. Carbomer 971P are the only pharmaceutical grades of polymer accepted for oral or mucosal contact products. Carbomers are synthetic high molecular weight polymers of acrylic acid that are crosslinked with either allylsucrose or allyl ethers of pentaerythritol. The compact sustained release tablet of the present invention comprises the granular phase covered with an extragranular phase which further contains a phase containing a lubricant and one or more hydrophilic polymers. The hydrophilic polymers may be the same as present in the granular phase or different. In one embodiment, the hydrophilic polymer present along with the lubricant is polyacrylic acid. Polyacrylic acid is generally known as Carbomer. A number of different carbomer grades are commercially available that vary in their molecular weight, degree of crosslinking polymer structure and residual components. These differences account for the specific rheological handling and use characteristics of each grade. Carbopol 971P has a viscosity in the range of 4000 to 11000 cP as a 0.5% w/v solution at 25° C. In one preferred embodiment, the gelling polymer used is carbomer having a viscosity in the range of 4000 to 11000 cP as a 0.5% solution at 25° C. The hydrophilic gelling polymer may be present in the range of 1.8% to 5%, preferably in the range of 2% to 3% by weight of the extragranular phase. The various grades of carbopol are differentiated with designation of numerical value like Carbopol 71G NF, Carbopol 971P NF, Carbopol 974P NF, Carbopol 934P NF. These polymers are manufactured in ethyl acetate which is a Ph. Eur./USP /ICH Class III solvent with GRAS status except for Carbopol 934P NF which is polymerized in benzene. The European Pharmacopeia has only one monograph which applies to Carbopol® polymers called “Carbomers”. The Japanese Pharmaceutical Excipients also has a single monograph called “Carboxyvinyl Polymer”. The United States Pharmacopeia/National Formulary has several monographs for different carbomer grades. The differentiation of monograph is based on viscosity characteristics like Type A—4,000-11,000; Type B—25,000-45,000; and Type C—40,000-60,000. Carbopol® 71G NF Polymer and Carbopol® 971P NF Polymer also known as Carbomer 941 are now categorized under Carbomer Homopolymer Type A; and Carbopol® 974P NF Polymer also known as Carbomer 934P is categorized as Carbomer Homopolymer Type B. In one preferred embodiment, Carbopol® 971P NF which is a lightly crosslinked polymer available in powder form is used. In another embodiment, Carbopol® 71G NF Polymer which is present in granular phase is used. Another grade suitable to be included as one of the hydrophilic polymer is Carbopol® 974P NF Polymer which is a highly crosslinked polymer available in powder form.

Other suitable examples of the hydrophilic polymer used in the compact sustained release tablet composition, include, but are not limited to, gums such as xanthan gum, guar gum, alginate and the like. Suitable examples of hydrophilic erodible polymers, include, but are not limited to, high viscosity grades of cellulose such as hydroxypropyl methyl cellulose K4M, K15M, K 100M. The K stands for the USP substitution type 2208 corresponding to a methoxy content of 19% to 24% and a hydroxypropoxy content of 4% to 12%. The high viscosity grades of the hydrophilic polymers are preferred having viscosity in the range of 2000 to 150000 cP. In a preferred embodiment, the hydroxypropyl methyl cellulose having viscosity grade of 80000 to 120000 mPa as measured at 20° C. in a 2% w/v aqueous solution is used.

The extragranular phase of the compact sustained release tablet of the present invention contains pharmaceutically acceptable tablet excipients such as for example, hydrophilic polymer that retard the release of the drug apart from lubricants, diluents or glidant or mixtures thereof. The hydrophilic polymers may be a gelling polymer or an erodible polymer or combination of the two. It is also possible to use Xanthan gum, a high molecular weight polysaccharide. It contains D-glucose and D-mannose as the dominant hexose units along with D-glucuronic acid. Its molecular weight is approximately 2×10⁶. Each xanthan gum repeat unit contains five sugar residues: two glucose, two mannose and one glucuronic acid. The polymer backbone consists of four β-D glucose units linked at the 1 and 4 positions and is therefore identical in structure to cellulose. Trisaccharide side chains on alternating anhydroglucose units distinguish xanthan from cellulose. The resulting stiff polymer chain may exist in solution as single double or triple helix which interacts with other xanthan gum molecules to form complex, loosely bound networks. It has a viscosity of 1200-1600 mPa for a 1% w/v aqueous solution at 25° C.

When the hydrophilic polymer used, it is present in the range of 45% to 95% by weight of the extragranular phase, more preferably in the range of 60% to 80% and most preferably in the range of 65% to 78% by weight of the extragranular phase. In one specific embodiment, the hydrophilic polymer is a mixture of high viscosity hydroxypropylmethyl cellulose and carboxy vinyl polymer. The high viscosity hydroxypropyl methyl cellulose is present in the range of 50% to 85% by weight of the extragranular phase and the polyacrylic acid (carbomer) is present in the range of 1.8% to 10% by weight of the extragranular phase. In another embodiment, the hydrophilic polymer is a mixture of xanthan gum and other hydrophilic polymer such as mixture of high viscosity hydroxypropylmethyl cellulose and carboxy vinyl polymer. It is also possible to use xanthan gum alone as a hydrophilic polymer in the extragranular phase.

The hydrophilic polymer is present in the range from about 10% to 40% by weight of the tablet, preferably, 15% to 30% by weight of the tablet.

Apart from the hydrophilic polymer, the extragranular phase comprises binders and diluents, lubricants. The binders used in the extragranular phase, include, but are not limited to polyvinyl alcohol, polyvinyl pyrrolidone, pregelatinised starch, sodium alginate, propylene glycol and mixtures thereof. The binders may be present in the range of 1% to 10% by weight of the extragranular phase, more preferably in the range of 1.5% to 8% and most preferably in the range of 2% to 6% by weight of the extragranular phase. The diluents used include but are not limited to lactose, lactitol, microcrystalline cellulose, calcium phosphate tribasic, dextrins, mannitol, and the like and mixtures thereof. The diluents may be present in a range of 1% to 25% by weight of the extragranular phase, more preferably in the range of 2% to 15% and most preferably in the range of 3% to 10% by weight of the extragranular phase.

Apart from these, the other excipients in the tablet are diluents, lubricants, binders and any other inactive excipients present in the tablet composition. The lubricants used within the scope of this invention include but are not limited to glyceryl behenate, talcum, waxes, hydrogenated castor oil, colloidal silicon dioxide, stearic acid, magnesium stearate and mixtures thereof. The lubricants may be present in the range of 1% to 40% by weight of the extragranular phase, more preferably in the range of 5% to 35% and most preferably in the range of 10% to 30% by weight of the extragranular phase. Without being bound to be bound by theory, the hydrophilic polymer present in the extragranular phase surrounds or covers the granular phase made up of divalproex or its pharmaceutically acceptable salt and hydrophobic polymer, and thereby controls the release of the divalproex or its pharmaceutically acceptable salt.

In one specific embodiment, the granular phase consists essentially of granular mixture of divalproex or its pharmaceutically acceptable salt and hydrophobic polymer and the extragranular phase containing hydrophilic polymer, are mixed intimately, either by further granulation using granulating fluid, or dry mixed and this admixture is further lubricated with lubricants, optionally diluents and additionally a fraction of the hydrophilic polymer and the blend is then compressed into a tablet. The compact sustained release tablet of the present invention does not contain any coating of water insoluble material such as for e.g. ethyl cellulose. The hydrophilic polymer used here may be a gelling polymer or an erodible polymer. Suitable examples, include, but are not limited to, high viscosity hydroxypropylmethyl cellulose, carboxy vinyl polymer, polyacrylic acid (carbomer), xanthan gum, guar gum, polyethylene oxide, alginic acid etc. In one specific embodiment, carbomers which are synthetic high molecular weight polymers of acrylic acid that are crosslinked with either allylsucrose or allyl ethers of pentaerythritol may be used. The amount of such hydrophilic polymer in the composition of the compact sustained release tablet ranges from 0.5% to 5%.

The compact sustained release tablet of the present invention may be prepared by any conventional process known in the art. In one aspect, the compact sustained release tablet of the present invention is prepared by the process of milling both the active agent and hydrophobic polymer separately in a comminuting mill and mixing together in a rapid mixer granulator. The mixture is granulated using a non-aqueous solvent and milled to get the appropriate size in a comminuting mill and then dried in a fluid bed drier to form the granular phase. The granular phase may also be prepared by granulation of active ingredient with a solution of ethyl cellulose in a suitable solvent. The extragranular phase is prepared by mixing the diluent, one or more hydrophilic polymers and other excipients together. The granules of granular phase are mixed with the extragranular phase in a mixer granulator. The mixture thus formed is again granulated using a hydro-alcoholic vehicle and the granules thus formed are reduced to appropriate size in a comminuting mill. The granules are dried in a fluid bed granulator at a temperature of 55° C. to 65° C. The granules are further blended with hydrophilic polymer and lubricants. The lubricated granules are then compressed into tablets and may further be film coated.

According to one embodiment of the present invention, a process for the preparation of a compact sustained release tablet of divalproex or its pharmaceutically acceptable salt, the process consisting essentially of:
a. preparing a granular phase consisting essentially of a granular mixture of divalproex or its pharmaceutically acceptable salt in amount ranging from about 700 mg to about 1500 mg of valproic acid and a hydrophobic polymer; and granulating with a solution of hydrophobic polymer in a non-aqueous solvent and drying the granular phase;
b. preparing an extragranular phase by
   i. mixing one or more hydrophilic polymers and pharmaceutically acceptable tablet excipients;
   ii. further granulating the granular mixture of step ‘a’, with one or more hydrophilic polymers and diluents; and drying the so formed granules;
   iii. blending the dried granules of step ‘ii’ with one or more hydrophilic polymers and lubricant; and
c. compressing the blend of step ‘iii’ into tablets.

In such embodiment, the water content of dried granular phase of step ‘a’ is about 0.5 weight. The weight ratio of divalproex or its pharmaceutically acceptable salt to hydrophobic polymer to is about 10. Particularly, the granular phase is prepared by a granulating fluid having non-aqueous solvents selected from methanol, dichloromethane, isopropyl alcohol, chloroform or mixture thereof. The granular phase is prepared under relative humidity of 40% to 45% and at a temperature of about 25° C. In one aspect of the process, the hydrophobic polymer is ethyl cellulose and is present in the range of about 5% to 10% of the weight of the tablet. The extragranular phase comprises a mixture of two or more hydrophilic polymer. The water content of the tablet is in the range of about 3% to 5% by weight.

According to another embodiment of the present invention, a process for the preparation of a compact sustained release tablet of divalproex or its pharmaceutically acceptable salt, the process consisting essentially of:
a. preparing a granular phase consisting essentially of a granular mixture of divalproex or its pharmaceutically acceptable salt in amount ranging from about 700 mg to about 1500 mg of valproic acid and a hydrophobic polymer; and granulating with a solution of hydrophobic polymer in a non-aqueous solvent and drying the granular phase;
b. preparing an extragranular phase by
   i. mixing one or more hydrophilic polymers and pharmaceutically acceptable tablet excipients;
   ii. further granulating the granular mixture of step ‘a’, with one or more hydrophilic polymers and diluents; and drying the so formed granules;
   iii. blending the dried granules of step ‘ii’ with one or more hydrophilic polymers and lubricant; and
c. compressing the blend of step ‘iii’ into tablets;
   wherein when the tablet is subjected to dissolution in 500 ml of 0.1N HCl for 45 min followed by 900 ml of pH 5.5 phosphate buffer with sodium lauryl sulphate using USP II dissolution apparatus and 100 rotation per minute, at 37° C.±0.5° C., releases divalproex or its pharmaceutically acceptable salts as follows:

| | % drug released | |
|---|---|---|
| Time in hours | Not Less than | Not more than |
| 3 | 10 | 20 |
| 9 | 40 | 50 |
| 12 | 50 | 60 |

According to another embodiment of the present invention, a process for the preparation of a compact sustained release tablet of divalproex or its pharmaceutically acceptable salt, the process consisting essentially of:
a. preparing a granular phase consisting essentially of a granular mixture of divalproex or its pharmaceutically acceptable salt in amount ranging from about 700 mg to about 1500 mg of valproic acid and a hydrophobic polymer; and granulating with a solution of hydrophobic polymer in a non-aqueous solvent and drying the granular phase;
b. preparing an extragranular phase by
   i. mixing one or more hydrophilic polymers and pharmaceutically acceptable tablet excipients;
   ii. further granulating the granular mixture of step ‘a’, with one or more hydrophilic polymers and diluents; and drying the so formed granules;
   iii. blending the dried granules of step ‘ii’ with one or more hydrophilic polymers and lubricant; and
c. compressing the blend of step ‘iii’ into tablets;
   wherein when subjected to oral administration, and when orally administered, with or without food, the ratio of mean AUC₀_{_}_{inf-fasted} to mean AUC₀_{_}_{inf-fed} is within the range of 0.9 to 1.1.

In one embodiment, the water content of the granular phase of the present invention is in the range of about 0.1% to 3%, preferably 0.5% to 2%. In another embodiment, the loss on drying of the granules is 0.5% or less. In another specific embodiment, the water content of the tablet of the present invention is in the range of about 3% to 5% by weight of tablet.

In an embodiment, the compact sustained release tablet of the present invention is administered orally in fed or fasted state. In another embodiment, the compact sustained release tablet of the present invention containing Divalproex 100 mg when administered orally in fed or fasted state, the ratio of mean AUC₀_{_}_{inf-fasted} to mean AUC₀_{_}_{inf-fed} is within the range of 0.9 to 1.1.

Also, the present invention provides a method of treating complex partial seizures, absence seizures; acute manic or mixed episodes associated with bipolar disorder or as a prophylaxis of migraine headaches; the method comprising orally administering compact sustained release tablet once a day, tablet comprising divalproex or its pharmaceutically acceptable salts as a sole active ingredient present in an amount equivalent to from about 700 mg to about 1500 mg of valproic acid and pharmaceutically acceptable tablet excipients; wherein weight ratio of pharmaceutically acceptable tablet excipients to divalproex and/or its salt is less than 1, and when orally administered, with or without food, the ratio of mean AUC₀_{_}_{inf-fasted} to mean AUC₀_{_}_{inf-fed} is within the range of 0.9 to 1.1.

Also, the present invention provides a method of treating complex partial seizures, absence seizures; acute manic or mixed episodes associated with bipolar disorder or as a prophylaxis of migraine headaches; the method comprising orally administering compact sustained release tablet once a day, the method comprising:
a hygroscopic active ingredient consisting of divalproex or its pharmaceutically acceptable salt and pharmaceutically acceptable tablet excipients wherein the tablet comprises
   a granular phase consisting essentially of granular mixture of divalproex or its pharmaceutically acceptable salts in an amount equivalent to from about 700 mg to about 1500 mg of valproic acid and a hydrophobic polymer; and
   an extragranular phase,
      wherein the weight ratio of pharmaceutical tablet excipients to divalproex and/or its salt is less than 1, wherein the sustained release tablets gradually release the divalproex over a period of 24 hrs. In one embodiment, the compact sustained release tablet is capsule shaped tablet and has dimensions as follows: Length ranging from 20 mm to 23 mm; Diameter ranging from of 11 mm to 14 mm and thickness ranging from 10 mm to 12 mm.

Also, the present invention provides an improvement in the method of administering divalproex or its pharmaceutically acceptable salt by orally administering once-daily one or multiple units of compact sustained release tablets containing divalproex or its pharmaceutically acceptable salt in an amount equivalent to 250 mg or 500 mg of valproic acid per tablet the improvement comprising orally administering to a patient in need thereof once-daily one or multiple units of compact sustained release tablet, each tablet comprising divalproex or its pharmaceutically acceptable salts in an amount equivalent to from about 700 mg to about 1500 mg of valproic acid, wherein the weight ratio of pharmaceutical tablet excipients to divalproex and/or its salt is less than 1 wherein the sustained release tablets gradually release the divalproex over a period of 24 h, whereby the number of tablets to be ingested by the patient is one or two. Below is the illustration of reduced number of tablets for various indications:

| | | | | Number of | Number of | |
|---|---|---|---|---|---|---|
| | | | Maximum | tablets of | compact | Reduction in |
| | Recommended | | allowable | Depakote ER | sustained | number of |
| Indication | dose | | dose | in prior art | release tablet | tablets |
| Mania | 25 | mg/kg/day | 1500 mg | 3 of 500 mg | 2 of 750 mg | From 3 to 2 |
| Complex | 15 | mg/kg/day | 900 mg | 2 of 500 mg | 1 of 1000 mg | From 2 to 1 |
| partial Seizures | | | | | | |
| Absence | 25 | mg/kg/day | 1500 mg | 3 of 500 mg | 2 of 750 mg | From 2 to 1 |
| Seizures | | | | | | |
| Migraine | 1000 | mg/day | 1000 mg | 2 of 500 mg | 1 of 1000 mg | From 2 to 1 |

In one specific embodiment, the present invention provides a compact sustained release tablet comprising divalproex or its pharmaceutically acceptable salt equivalent to 1250 mg of valproic acid in one unit of the compact sustained release tablet, more preferably, equivalent to 1000 mg per unit of valproic acid in one unit of the compact sustained release tablet. In yet another embodiment, the present invention also provides improvement in the method of administering divalproex or its pharmaceutically acceptable salt equivalent to 750 mg of valproic acid in one unit of the compact sustained release tablet, wherein when orally administered, it provides equivalent oral bioavailability irrespective of whether the tablet is administered with or without food. Also, the present invention provides method of treating different types of epilepsy like complex partial seizures, absence seizures; acute manic or mixed episodes associated with bipolar disorder; and for prophylaxis of migraine headaches by orally administering a compact sustained release tablet comprising divalproex or its salts, wherein the number of units of the tablets to be ingested by the patient is reduced as compared to the prior art tablets that are available under the brand name of Depakote ER™, available as divalproex sodium having 250 mg and 500 mg of valproic acid. The improvement can be illustrated as follows: For a patient weighing 30 kg, is prescribed a dose of 55 mg/kg/day of divalproex sodium equivalent to 1650 mg of valproic acid. In this case, according to the present invention, one unit of compact sustained release tablet of 1000 mg and 1 unit of compact sustained release tablet of 750 mg, would achieve the dose. In contrast, according to the prior art, the patient would require to ingest three units of tablets of 500 mg and one tablet of 250 mg of the commercially marketed product Depakote ER™ which achieves the prescribed dose of 1750 mg instead of 1650 mg. Thus, the present invention achieves the dose by administering two units of the tablets as against four units of the tablets Depakote ER™. Similarly, for a patient weighing 60 kg who is prescribed valproic acid as divalproex sodium, at a dose of 55 mg/kg/day for the treatment of mania, daily dose to be achieved is 3300 mg. In this case, according to the present invention, two units of compact sustained release tablet of 1250 mg and 1 unit of compact sustained release tablet of 1000 mg, would achieve the dose of 3500 mg. In contrast, according to the prior art, the patient would require to ingest seven units of tablets of 500 mg of the commercially marketed product Depakote ER™ which achieves the prescribed dose of 3500 mg instead of 3300 mg. Thus, the present invention achieves the dose by administering three units of the tablets as against seven units of the tablets of Depakote ER®^{.} Each unit of the compact sustained release tablet contains divalproex or its salt, expressed as valproic acid, in amounts of 700 mg, 750 mg, 800 mg, 850 mg, 900 mg, 950 mg, 1000 mg, 1100 mg, 1200 mg, 1250 mg, 1300 mg, 1400 mg or 1500 mg per unit tablet. In one preferred embodiment, it may be divalproex in amounts equivalent to 750 mg, 1000 mg, 1250 mg, or 1500 mg of valproic acid. In an embodiment, the number of units of compact sustained release tablet of the present invention administered to a patient is reduced by at least 50% as compared to when same dose of prior art formulation Depakote ER is administered. For example, the compact sustained release tablet of the present invention can be given as follows in reduced number of pills compared to the what is existing in the prior art known under the brand name Depakote ER. Depakote ER is available in only two strengths, which contain 250 mg and 500 mg divalproex.

While the present invention is disclosed generally above, additional aspects are further discussed and illustrated with reference to the examples below. However, the examples are presented merely to illustrate the invention and should not be considered as limitations thereto.

The compact sustained release tablet of the present invention is illustrated in the example below:

### EXAMPLE 1

A granular phase was prepared as per the following formula:

TABLE 1
Composition details of granular phase

| | | % of |
|---|---|---|
| Ingredient | | granular |
| Category | Name | phase |
| Active ingredient | Divalproex sodium | 90% |
| Hydrophobic polymer | Ethyl cellulose | 10% |
| Organic solvent | Isopropyl | q.s. |
| | alcohol:Dichloromethane (1:1) | |

Preparation of Granular Phase

Divalproex sodium was milled through an appropriately sized screen in a comminuting mill. Ethyl cellulose was sifted through appropriate sieve and milled divalproex sodium and sifted ethyl cellulose was mixed in a rapid mixer granulator. The mixture was granulated using ethyl cellulose dissolved in isopropyl alcohol methylene chloride (1:1) by mixing the wet mass and the granules were milled in a comminuting mill and then dried in a fluid bed drier and further air dried to obtain granular mixture. Granules were sifted through appropriate sieve. This formed the granular phase.

An extragranular phase was prepared as per the following formula:

TABLE 2
Composition details of extragranular phase

| | | % of |
|---|---|---|
| Ingredient | | extragranular |
| Category | Name | phase |
| Hydrophilic | Hydroxypropylmethyl cellulose | 60-75% |
| polymer | (High viscosity grade) | |
| Binder | Polyvinyl pyrrolidone | 2-6% |
| Gelling polymer | Polyacrylic acid | 0.1-2.5% |
| Diluent | Micro crystalline cellulose/ | 0.1 to 5% |
| | Anhydrous lactose | |
| Lubricants | Talc | 1-4% |
| | Magnesium stearate | 5-15% |
| Glidant | Silicon dioxide | 5-15% |
| Film coating | Hydroxypropyl methyl | 2-3% |
| | cellulose based coating | |

Preparation of extragranular phase: Anhydrous lactose, hydroxypropylmethyl cellulose, polyvinyl pyrrolidone and carbomer were sifted through appropriate sieve.

Admixture of Granular and Extragranular Phase and Preparation of Granules

The granules of the granular phase weighing 1178 mg were mixed with the extragranular phase weighing 622 mg in a rapid mixer granulator. The material was granulated using a mixture of water and isopropyl alcohol and the granules thus formed were milled through a 10 mm screen using a comminuting mill. The granules were dried in a fluid bed granulator at a temperature of 55° C. to 65° C. The dried granules were sifted through #18 sieve.

Blending, Tablet Compression and Film Coating

The granules were further blended with talc, polyacrylic acid and magnesium stearate and lubricated with silicon dioxide. The lubricated blend of granules of the granular phase and extragranular phase in suitable amounts can be taken and compressed into a tablet containing divalproex sodium equivalent to 1000 mg of valproic acid.

The tablets may further be coated with a non-functional coating for example having a coating composition with a low viscosity grade water soluble polymer such as hydroxypropyl methyl cellulose.

Following the method of the above example tablets of different strengths for example 700 mg, 750 mg, 800 mg, 850 mg, 900 mg, 950 mg, 1000 mg, 1100 mg, 1200 mg, 1250 mg, 1300 mg, 1400 mg or 1500 mg are prepared.

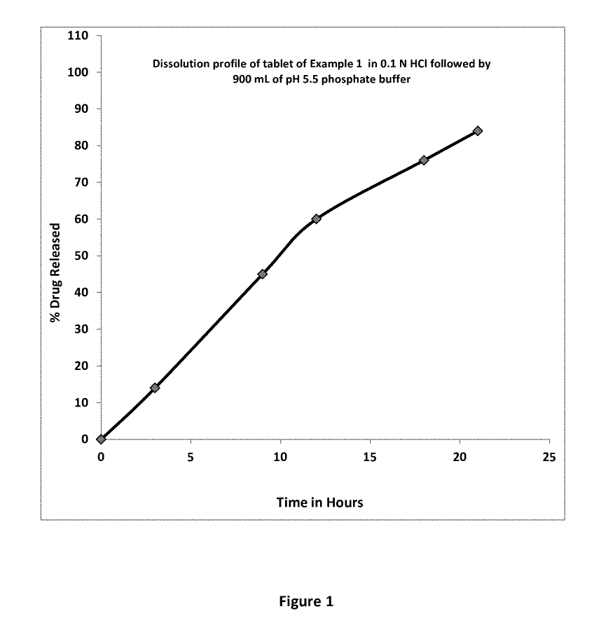

The coated tablet composition prepared as per the example 1 was subjected to dissolution in 500 ml of 0.1 N hydrochloric acid for first 45 min, followed by 900 ml of 0.05M phosphate buffer containing 75 mM sodium lauryl sulphate at pH 5.5, Type 2 USP dissolution apparatus, operating at 37° C. with a paddle rotating speed of 100 rpm. The results of the dissolution study are as depicted in FIG. 1. The dissolution data is as given in the table below:

TABLE 3
Dissolution profile of tablet composition prepared as per example 1 in
0.1N HCl followed by 900 mL of pH 5.5 phosphate buffer
Dissolution profile of tablet composition prepared as per example 1

| Time in hours | % drug released |
|---|---|
| 0 | 0 |
| 1.5 | 5 |
| 3 | 14 |
| 6 | 31 |
| 9 | 45 |
| 12 | 60 |
| 15 | 67 |
| 18 | 76 |
| 21 | 84 |
| 24 | 91 |

From the dissolution data and the graph of FIG. 1, it is apparent that the divalproex sodium sustained release tablets showed a zero order release profile. There was low incidence of tablet defects. The tablets did not rupture or burst to release the water soluble drug all at once.

### EXAMPLE 2

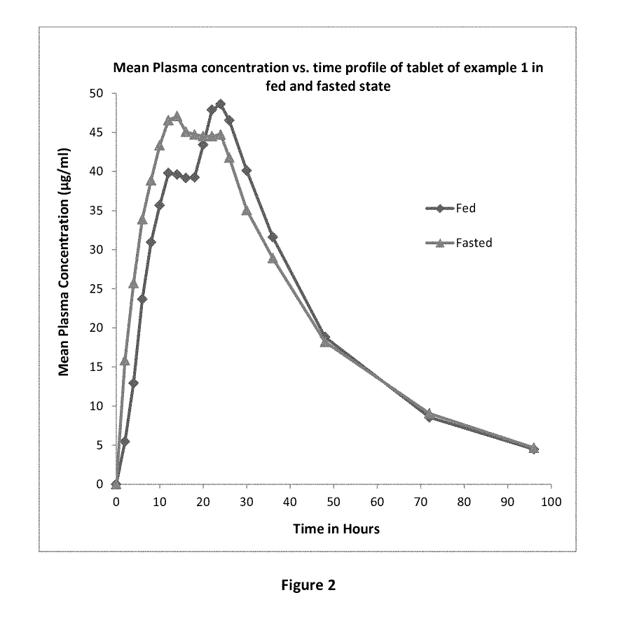

Tablets prepared as per Example 1, one tablet containing divalproex sodium equivalent of 1000 mg of valproic acid was subjected to oral bioavailability studies, two parallel groups each of 15 healthy volunteers. Divalproex 1000 mg tablet prepared as per the example 1 was administered to first group in fasted state and was administered to the other group 30 minutes after a high calorie high fat breakfast (fed state). Post-dose blood samples were collected at 2, 4, 6, 8, 10, 12, 14, 16, 18, 20, 22, 24, 26, 30, 36, 48, 72 and 96 hours and analysed for valproic acid plasma concentration. The results are given in FIG. 2 and Table 4.

TABLE 4
Pharmacokinetic study of Divalproex 1000 mg in fasting and fed state

| | Fasted State | | | Fed State | |
|---|---|---|---|---|---|
| | Mean | | | Mean | |
| | Plasma | % | | Plasma | % |
| Parameters | Levels | CV | Parameters | Levels | CV |
| AUC₀_t-fasted | 1999.7 | 30.7 | AUC₀_t-fed | 1949.1 | 30.3 |
| AUC₀_inf-fasted | 2155.5 | 33.2 | AUC₀_inf-fed | 2081 | 34 |
| C_{max-fasted} | 51.7 | 23.6 | C_{max-fed} | 58 | 19 |
| T_{max-fasted} | 16 | 42.9 | T_{max-fed} | 18 | 34.9 |

It may be concluded that when compact sustained release tablets of the present invention are orally administered with or without food the ratio of mean AUC₀_{_}_{inf-fasted} to mean AUC₀_{_}_{inf-fed} is within the range 0.9 to 1.1.

1. A compact sustained release tablet comprising divalproex or its pharmaceutically acceptable salts as a sole active ingredient is present in an amount equivalent to from about 700 mg to about 1500 mg of valproic acid and pharmaceutically acceptable tablet excipients; wherein weight ratio of pharmaceutically acceptable tablet excipients to divalproex or its salt is less than 1, the tablet is suitable for once a day administration and when orally administered, with or without food, the ratio of mean AUC₀_{_}_{inf-fasted} to mean AUC₀_{_}_{inf-fed} is within the range of 0.9 to 1.1.

2. A compact sustained release tablet as claimed in claim 1, wherein the tablet comprises
a granular phase consisting essentially of a granulated mixture of divalproex or its pharmaceutically acceptable salts in an amount equivalent to from about 700 mg to about 1500 mg of valproic acid and a hydrophobic polymer; and
an extragranular phase.

3. A compact sustained release tablet as claimed in claim 2, wherein the weight ratio of the divalproex or its pharmaceutically acceptable salt to hydrophobic polymer is about 10.

4. A compact sustained release tablet as claimed in claim 2, wherein the extragranular phase comprises a mixture of two or more hydrophilic polymer.

5. A compact sustained release tablet as claimed in claim 2, wherein the hydrophobic polymer is present in the range of 5% to 15% by weight of the tablet.

6. A compact sustained release tablet as claimed in claim 2, wherein the hydrophilic polymer is present in the range of 15% to 30% by weight of the tablet.

7. A compact sustained release tablet as claimed in claim 2, wherein the hydrophobic and the hydrophilic polymers are present in the range of 30% to 45% by weight of the tablet.

8. A compact sustained release tablet as claimed in claim 7, wherein the hydrophilic polymer is selected from hydroxypropyl methyl cellulose having a molecular weight of about 10000-1500000; and polyacrylic acid having a molecular weight in a range of about 700000 to 4000000, having viscosity in a range of about 3000 to 60000 cps.

9. A compact sustained release tablet as claimed in claim 8, wherein hydroxypropyl methyl cellulose is present in a range of about 20% to 40% by weight of the tablet and polyacrylic acids is present in the range of 0.5% to 1.0% by weight of the tablet.

10. A compact sustained release tablet as claimed in claim 1, wherein when the tablet is subjected to dissolution in 500 ml of 0.1N hydrochloric acid for 45 min followed by 900 ml of pH 5.5 phosphate buffer with sodium lauryl sulphate using USP II dissolution apparatus rotating at a speed of 100 rotations per minute, releases divalproex or its pharmaceutically acceptable salts as follows:

| | % divalproex released | |
|---|---|---|
| Time in hours | Not Less than | Not more than |
| 3 | 10 | 20 |
| 9 | 40 | 50 |
| 12 | 50 | 60 |

11. A compact sustained release tablet as claimed in claim 2, wherein the water content of dried granular phase is less than 0.5% by weight of the granular phase.

12. A compact sustained release tablet as claimed in claim 1, wherein the water content of the tablet is in the range of about 3% to 5% by weight.
